(19) United States
(12) Patent Application Publication
(10) Pub. No.: US 2024/0358539 A1
(43) Pub. Date: Oct. 31, 2024

(54) Urinary Catheter Privacy Bag for Wheelchairs

(71) Applicant: Taylar Gallup, Cheshire, MA (US)

(72) Inventor: Taylar Gallup, Cheshire, MA (US)

(21) Appl. No.: 18/646,743

(22) Filed: Apr. 25, 2024

Related U.S. Application Data
(60) Provisional application No. 63/461,662, filed on Apr. 25, 2023.

(51) Int. Cl. A61F 5/44

(52) U.S. Cl. CPC A61F 5/44

(57) ABSTRACT

A novel container designed to enhance the utility and functionality of wheelchairs by providing a discrete, sanitary, and easily accessible storage solution for urinary catheter drainage bags. This innovative container can mitigate common issues associated with traditional catheter bag placement, such as infections, privacy concerns, and the risk of traumatic catheter removals. The container can be attached to various wheelchair models allows for the independence and dignity of users by providing simplified, private access to the catheter bag without requiring assistance. Additionally, the design ensures the catheter bag remains at an optimal position to prevent backflow and reduce the risk of catheter-associated urinary tract infections (CAUTIs).

### CROSS-REFERENCES TO RELATED PATENT APPLICATIONS

This application claims the benefit of U.S. Provisional Application No. 63/461,662, filed on Apr. 25, 2023, the entire contents of which is incorporated herein by reference.

### STATEMENT REGARDING FEDERALLY SPONSORED RESEARCH OR DEVELOPMENT

### Not Applicable.

### REFERENCE TO SEQUENCE LISTING, A TABLE, OR A COMPUTER PROGRAM LISTING COMPACT DISK APPENDIX

Not Applicable.

### BACKGROUND

Embodiments of the inventive subject matter relate to medical devices and more specifically to devices that are effective in securing urinary catheters to chairs and wheelchairs to maintain safety and hygiene. Embodiments include systems, apparatuses and methods for transporting a urinary drainage bag on a wheelchair that allows independence of the individual, decreases risk of infection, and decreases risk of injury.

The prior art includes a number of various solutions for the attachment of urinary catheter bags to wheelchairs and other devices. A common healthcare related infection contracted by patients with urinary catheters is catheter-associated urinary tract infection (CAUTI)

Nearly 40% of all infections acquired in hospitals are urinary tract infections related to catheter use. CAUTIs can lead to more serious complications such as sepsis and endocarditis, and it is estimated that over 13,000 deaths each year are associated with UTIs. They also increase patient discomfort and almost universally require antibiotic drug therapy, ultimately leading to unnecessary healthcare costs. Moreover, because of increased Medicare and Medicaid scrutiny, CAUTI prevention has become a top priority for hospitals and other healthcare providers.

CAUTI can occur for many reasons, including the patient's lack of hygiene, transmission by health-care personnel handling catheter insertion, and simply a lack of sterility of catheter tubing during extended catheterization. Additionally, one of the primary causes of CAUTI is lack of proper drainage and subsequent urine backflow combined with excessive manipulation of the catheter and its attendant drainage bag during patient transport. Empirical evidence has shown that the failure to have proper tubing and drainage bag placement significantly increases the risk of CAUTI. In order to assure proper drainage and reduce the instance of CAUTI, the catheter collection tubing and drainage bag should always remain at an elevation below the patient's bladder and the collection tubing should always remain above the drainage bag.

Wheelchair transport is one of the most common situations where improper catheter tubing and drainage bag placement and excessive manipulation occur. A common current practice during wheelchair transport is to simply place the catheter drainage bag on the patient's lap. This practice presents three primary problems: (1) the catheter tubing and drainage bag are at an elevation above the patient's bladder which leads to the aforementioned improper urine drainage; (2) the tubing and drainage bag are susceptible to getting tangled in the wheelchair frame and/or wheels, which leads to excessive manipulation of the catheter, increasing the possibility of infection and injury to the patient, and; (3) it is unsightly and often embarrassing for a patient to have to hold the drainage bag on his/her lap. Thus, there exists a need for an economical and effective device for adequately supporting the catheter tubing and drainage bag during wheelchair transport while also greatly improving aesthetics and ease of use.

A significant number of individuals require use of a urinary catheter to manage urinary incontinence or retention each year. Per Grand View Research, the global market size of global urinary catheters was estimated at USD 5.03 billion in 2021 and was expected to reach USD 5.3 billion in 2022. Many individuals who require the use of a urinary catheter also require the use of a wheelchair for safe and independent functional mobility, especially in hospitals and long-term care settings. There are roughly 50,000 residents in nursing homes with catheters at any given time, and 70% of residents in nursing homes spend some of their day in a wheelchair. While urinary catheters are necessary for the health and everyday functioning of many individuals, they do not come without risks and threats to the safety of these same people. The insertion of a urinary catheter increases the ease of entry for microbes into the body, placing individuals at a high risk for catheter-associated urinary tract infections (CAUTIs). The current lack of a functional place to store a urinary drainage bag and tubing on a wheelchair often results in these bags dragging on or touching the floor, significantly increasing the risk for infection. Individuals utilizing indwelling urinary catheters are also at risk for traumatic removal, where the tubing is removed prior to deflating the balloon located inside of the bladder. The balloon is designed to keep the tubing in place during use.

The other current method of placement of urinary drainage bags on wheelchairs results in the bag being attached to the wheelchair. If an individual is transferred from their wheelchair before freeing the drainage bag, a traumatic removal is likely to occur. Current solutions on the market require the assistance of another individual to obtain and place the drainage bag into a narrow, vertically hanging privacy bag behind the wheelchair, significantly decreasing the independence and quality of life of the individual. Utilizing a privacy bag is taxing, even for caregivers, and therefore is often not used. Not using a privacy bag places the individual at risk for infection, traumatic removal, decreased independence, emotional distress, and embarrassment. Individuals with functional issues attempting to reach and bend forward to obtain the drainage bag attached underneath the wheelchair are at a high risk of falling out of their wheelchair and sustaining other injuries.

### SUMMARY

The illustrative embodiments provide apparatuses and methods for a container to keep the urinary drainage bag off of the floor (decreasing risk of infection) in a container that provides privacy and is easily accessible by the individual thereby providing increased independence and decreasing risk of falling. The container allows for the drainage bag to be easily removed from the container thereby decreasing the risk of traumatic removal from accidental transfers prior to obtaining the drainage bag.

One implementation of the claimed subject matter provides a method for transporting a urinary drainage bag on a wheelchair that allows independence of the individual, decreases risk of infection, and decreases risk of injury. Many of the embodiments provide a place for a urinary catheter drainage bag to be used with a wheelchair without compromising the person's independence, safety, or dignity. Currently existing wheelchairs do not have appropriate facilities to hang/place/store a catheter bag without the catheter bag dragging on the floor. This practice can put the patient at risk for CAUTIs. The problem is further exacerbated because without help from another person the patient will have difficulty draining the bag or obtaining the bag in order to transfer to other surfaces (i.e., bed, recliner chair, toilet, car). This limits their independence and quality of life. Current solutions for storing a catheter bag on a wheelchair also put an individual at risk of having a traumatic removal of the catheter, which can cause irreversible damage to the bladder and urinary tract.

In one embodiment, a wheelchair accessory for managing a urinary catheter drainage bag is provided including a container designed to house at least one urinary catheter drainage bag and at least one connector for removably attaching the container to a wheelchair. The container is positioned to maintain the drainage bag at an elevation below the user's bladder when seated in the wheelchair. In another embodiment, the container is positioned in front of the wheelchair to facilitate user access. In another embodiment, the container includes a closure mechanism to secure the drainage bag within the container. In other embodiments, the closure mechanism is selected from the group consisting of a clasp, magnet, hook and loop closure, button and hole closure, zipper, and snaps. In other embodiments, the container is made from materials selected from the group consisting of cloth, canvas, plastic, and composite materials. In other embodiments, wherein the connector allows for quick detachment of the container for emergency or routine cleaning. Some embodiments include a plurality of containers for managing multiple drainage bags.

### DETAILED DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

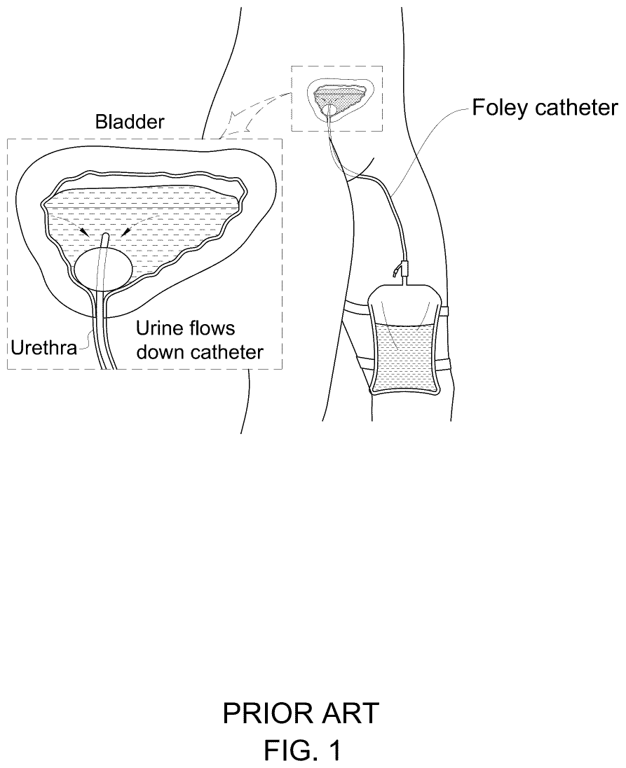

FIG. 1 is of the prior art and shows the balloon mechanism involved in holding the foley catheter tubing in place. An individual can choose to use a standard urinary drainage bag, or a leg bag. A leg bag is usually used when going out in the community as it can be hidden underneath pants, but it does not hold as much as a standard bag and is not recommended to be used overnight.

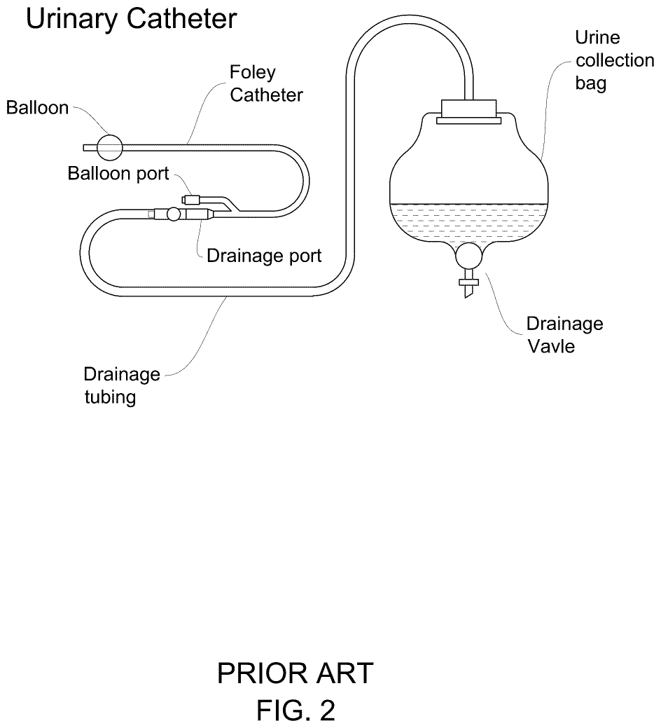

FIG. 2 is of the prior art and shows all aspects of a urinary catheter and a standard urinary drainage bag. The blue mechanism seen on top of the bag is a hook which can be used to hang the bag from a chair or hospital bed.

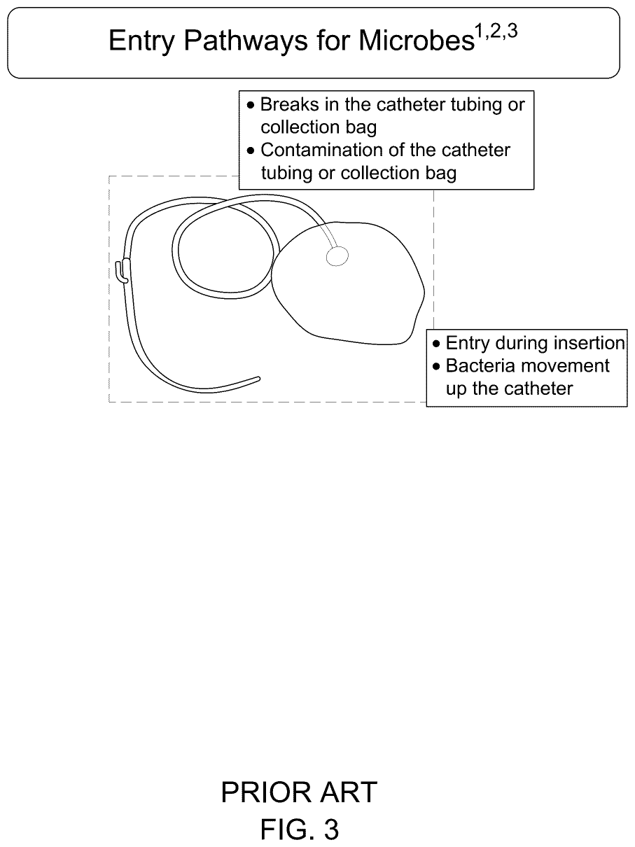

FIG. 3 is a picture of a wheelchair showing the container of the instant invention from the front left hand side view attached to a wheelchair.

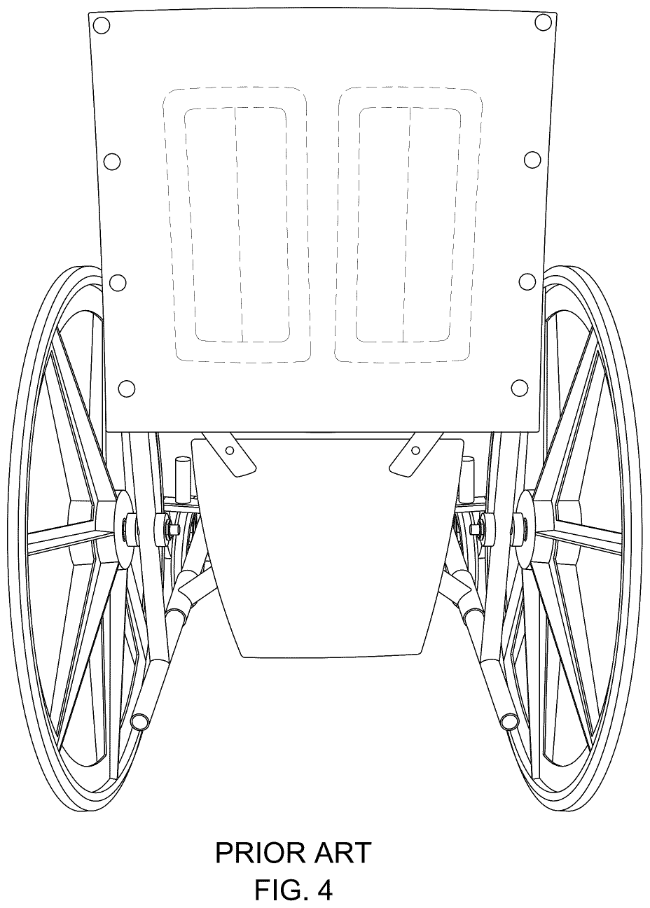

FIG. 4 shows a typical privacy bag;

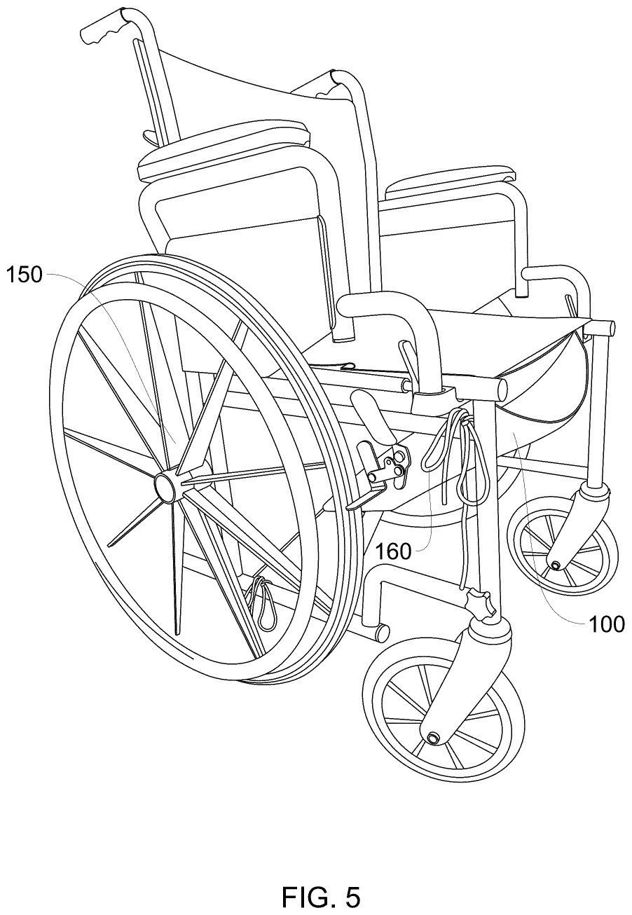

FIG. 5 shows the prior art entry pathways for microbes that cause urinary infections; and

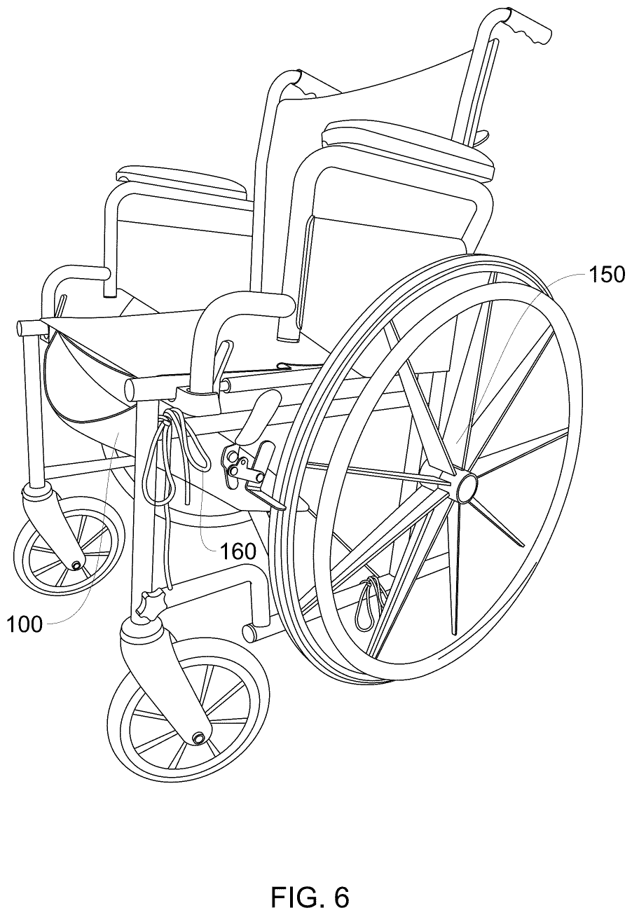

FIG. 6 is a picture of a wheelchair showing the container of the instant invention from the front view attached to a wheelchair.

### DETAILED DESCRIPTION OF THE EMBODIMENTS

While various aspects and features of certain embodiments have been summarized above, the following detailed description illustrates exemplary embodiments in further detail to enable one skilled in the art to practice such embodiments. The described examples are provided for illustrative purposes and are not intended to limit the scope of the claimed subject matter.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the claimed subject matter may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As the inventive subject matter is susceptible to embodiments of many different forms, it is intended that the present disclosure be considered as an example of the principles of the claimed subject matter and not intended to limit the scope of the claimed subject matter to the specific embodiments shown and described.

Many of the embodiments include a container to hold a urinary catheter when used with a wheelchair. Urinary catheters are commonly used medical devices that assist with draining the bladder. They are used for managing urinary incontinence, urinary retention (the inability to empty your bladder when needed), or for other medical conditions, such as spinal cord injuries, multiple sclerosis, or dementia. There are several types of catheters, including indwelling catheters, external catheters, and intermittent catheters.

One type of indwelling catheter is the foley catheter. The foley catheter is inserted through the urethra and is attached to a tube that drains urine into a bag outside of the body. A small balloon is inflated at the end of the tube inside of the bladder to prevent the tube from coming out and is deflated before removing. These catheters are used for extended periods of time and should be removed and replaced every 3 months. Foley catheters are often used by individuals who are unable to manage a catheter on their own. Another type of indwelling catheter is a suprapubic catheter, which is placed through a small opening in the lower abdomen rather than through the urethra. Individuals changing suprapubic catheters must hold a special certification to complete. An external catheter involves a device placed outside of the body such as a condom catheter for men or a PureWick® for women. These are used primarily for urinary incontinence. An intermittent catheter or straight catheter is used multiple times a day and is inserted through the urethra and into the bladder to drain the bladder and it is removed immediately after voiding. Many people requiring long term use of a catheter require an indwelling catheter due to limitations in functional abilities, such as decreased fine motor coordination, strength, and cognition. Others prefer to use an indwelling catheter for convenience purposes, as well as to limit discomfort experienced with continued insertion and removal required for straight catheterizing. The present embodiments are specifically designed to hold the drainage bag for an indwelling catheter.

A significant number of individuals have a need to use a urinary catheter to manage urinary incontinence or retention. Many individuals are required to use a urinary catheter and also require the use of a wheelchair for safe and independent functional mobility. This is especially true in hospitals and long-term care settings. Currently the nursing industries estimate that there are approximately 50,000 residents in nursing homes with catheters at any given time, and 70% of residents in nursing homes spend a significant amount of time each day in a wheelchair. While urinary catheters are necessary for the health and function of patients in health care facilities, they do not come without risks and threats to the safety. The insertion of a urinary catheter increases the ease of entry for microbes into the body, placing individuals at a high risk for catheter-associated urinary tract infections (CAUTIs). The current lack of functional placement for the urinary drainage bag on a wheelchair often results in these bags dragging on or touching the floor, significantly increasing the risk for infection. Individuals utilizing indwelling urinary catheters are also at risk for traumatic removal, where the tubing is removed prior to deflating the balloon located inside of the bladder that helps to keep the tubing in place. The current method of placement of urinary drainage bags on wheelchairs is semifixed, and if an individual attempts to transfer from their wheelchair before obtaining the drainage bag, a traumatic removal is likely to occur. Current solutions on the market require the assistance of another individual to obtain and place the drainage bag into a narrow, vertically hanging bag behind the wheelchair, significantly decreasing the independence and quality of life of the individual. Utilizing this bag is taxing, even for caregivers, and therefore is often forgone, placing the individual again at risk for infection, traumatic removal, decreased independence, and emotional distress/embarrassment. Individuals with functional deficits attempting to reach and bend forward to obtain the drainage bag attached underneath the wheelchair are at a high risk of falling out of their wheelchair and sustaining other injuries. The invented device proposed in this paper keeps the urinary drainage bag off of the floor (decreasing risk of infection) in a contained bag that provides privacy, is easily accessible by the individual (providing increased independence and decreasing risk of falls) and allows for the drainage bag to easily slide out of in case of a transfer prior to obtaining the drainage bag (decreasing risk of traumatic removal).

Many of the embodiments provide a place for a urinary catheter drainage bag to hang on a wheelchair in a position that helps in not compromising the person's independence, safety, or dignity. Currently, wheelchairs do not provide a convenient and sanitary place to hang/place/store a catheter bag without the bag dragging on the floor leading to a risk in infection by increasing the possibility of transmitting a microbe that can cause infection/CAUTIs. The problem of not having a convenient place to locate the collection bag means that most often the patient will require help from another person (limiting the patient's independence and quality of life.) Current solutions for storing a catheter bag on a wheelchair also put an individual at risk of having a traumatic removal of the catheter, an adverse event. This type of event can cause irreversible damage to the bladder and urinary tract. Prior art wheelchair privacy bags for catheters have 2 straps with the bag positioned to hang vertically behind the wheelchair.

In the described embodiments, the container is a separate container that is designed to hold the catheter drainage bag and it has one or more attachments thereby allowing the container to be hung at a diagonal angle and in the front of the wheelchair, directly below the patient. This allows the patient to place and remove the urinary drainage/catheter bag from the container attached to the wheelchair on their own. The straps have a universal connection method or connectors so that it is usable with all types and sized wheelchairs. The container can also have a closure mechanism. The closure mechanism will allow the container to easily close at top for increased privacy but can easily be undone so that if the person transfers from the wheelchair prior to obtaining the bag, that the closure mechanism will easily come undone, and the bag will be able to slide out of the container to limit risk of traumatic removal. The closure mechanism can be selected from the group comprising of a clasp, magnet, hook and loop closure such as Velcro®, button and hole closure, zipper or snaps.

Embodiments can be used with a wheelchair and for individuals who use wheelchairs and have a urinary catheter. The container can be used at home and in the community, but also in nursing homes and hospitals, where wheelchair and urinary catheter usage is high. Most institutions such as a nursing homes and hospitals currently use wheelchair privacy bags that are purchased in bulk and are often used by one patient throughout their stay and then discarded. The instant invention container is designed to be reused and can be laundered to decrease waste and costs, maximizing sustainability. The container can be made from cloth, canvas, plastic or plastic films or composites.

Furthermore, many embodiments provide straps that are placed in locations to fit a wheelchair frame and allow the container to hang at an optimal, diagonal angle in the front of the wheelchair. Other angles may also be implemented in other embodiments. Also in these embodiments, the size of container is optimal for easy storage of a urinary catheter drainage bag. The container allows for easy laundering and allows the catheter bag to easily be removed from the container in case an individual forgets to remove it before walking/transferring out of wheelchair.

Referring now to the FIGS. 1-6 and more specifically FIG. 1 there is shown the prior art. FIG. 1 shows the balloon mechanism involved in holding the foley catheter tubing in place. An individual can choose to use a standard urinary drainage bag, or a leg bag. A leg bag is usually used when going out in the community as it can be hidden underneath pants, but it does not hold as much as a standard bag and is not recommended to be used overnight.

FIG. 2 is of the prior art and shows all aspects of a urinary catheter and a standard urinary drainage bag. The blue mechanism seen on top of the bag is a hook which can be used to hang the bag from a chair or hospital bed.

FIG. 3 shows the prior art entry pathways for microbes that cause urinary infections.

FIG. 4 shows the prior art typical privacy bag.

FIG. 5 is a picture of a wheelchair showing the container 100 of the instant invention from the front left hand side view attached to a wheel chair150.

FIG. 6 is a picture of a wheelchair showing the container 100 of the instant invention from the front view attached to a wheelchair 150. The container has connectors 160 that allows the container 100 to be removably attached to the wheelchair 150.

The above embodiments can be used with a wheelchair and include a container, at least one connector. The container is configured to hold at least one urinary catheter bag and the container is removably attached to the wheelchair using the at least one connector.

The container allows the bag connected to a urinary catheter to be held in a discrete and easily removable location so that a patient does not need the assistance of a health care worker or another individual to aid.

Some embodiments include an integrated hygiene module within or connected to the container that allows UV-C light for sterilization or a compartment with pre-moistened disinfecting wipes. This module can help in maintaining sterility of the catheter and the drainage bag, potentially reducing the risk of CAUTIs further by ensuring the cleanliness of the catheter interface.

Some of the embodiments are constructed of a modular design that can be adapted for different wheelchair designs or user needs, such as use with a mobility scooter. Modules could include different attachment mechanisms, varying bag sizes, or features such as additional pockets for personal items or medical supplies.

Some of the embodiments include a temperature control system within the container to maintain an optimal temperature environment for the urinary drainage bag. This could be particularly useful in very cold or hot climates where temperature extremes could affect the function of the catheter or the comfort of the user.

Some of the embodiments include smart technology integration such as the use of smart sensors within the container that can detect the fill level of the urinary drainage bag and alert the user via a mobile app or a small display on the wheelchair. This technology could also track the time since the last emptying to aid in regular maintenance.

Some of the embodiments include options for aesthetic customization of the container's exterior appearance. For example, customizable covers may be used with the container. These covers can be used in various colors and designs to match the personal style or the color scheme of the wheelchair. This can help in enhancing the dignity and personal expression of the user.

Some of the embodiments include expandable containers to accommodate different sizes of urinary drainage bags, adapting to the user's needs throughout the day or for longer trips where a larger bag might be necessary.

Some of the embodiments include a locking mechanism for the container so that the urinary drainage bag can be secured against unintended removal, providing safety against accidental openings when navigating over uneven surfaces or during transfers.

Some of the embodiments include waterproof, water resistant and/or odor-control materials to help ensure that the container remains hygienic and discreet in public settings.

Some of the embodiments include ergonomic accessibility features such as ergonomic design features that enhance ease of access, such as handles or grips that are easy to reach and operate by individuals with limited dexterity, improving the independence of the user.

Some of the embodiments may also include an educational interface that could be used with the container. It could be a physical interface such as cards or it could be a digital app that provides users and caregivers with instructions on proper care and maintenance of both the catheter and the privacy bag system, potentially reducing the risk of misuse and enhancing the training process.

Since many modifications, variations, and changes in detail can be made to the described embodiments of the invention, it is intended that all matters in the foregoing description and shown in the accompanying drawings be interpreted as illustrative and not in a limiting sense.

Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While the inventive subject matter has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiment. It is to be understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

What is claimed is:

1. A wheelchair accessory for managing a urinary catheter drainage bag, comprising:
a container designed to house at least one urinary catheter drainage bag;
at least one connector for removably attaching the container to a wheelchair;
wherein the container is positioned to maintain the drainage bag at an elevation below the user's bladder when seated in the wheelchair.

2. The wheelchair accessory of claim 1, wherein the container is positioned in front of the wheelchair to facilitate user access.

3. The wheelchair accessory of claim 1, wherein the container includes a closure mechanism to secure the drainage bag within the container.

4. The wheelchair accessory of claim 3, wherein the closure mechanism is selected from the group consisting of a clasp, magnet, hook and loop closure, button and hole closure, zipper, and snaps.

5. The wheelchair accessory of claim 1, wherein the container is made from materials selected from the group consisting of cloth, canvas, plastic, and composite materials.

6. The wheelchair accessory of claim 1, wherein the connector allows for quick detachment of the container for emergency or routine cleaning.

7. The wheelchair accessory of claim 1, further comprising a plurality of containers for managing multiple drainage bags.

8. The wheelchair accessory of claim 1, wherein the container is adaptable to different sizes and types of drainage bags.

9. The wheelchair accessory of claim 1, wherein the container is designed to prevent accidental detachment or spillage of the contents of the drainage bag.

10. A method for reducing the risk of catheter-associated urinary tract infections (CAUTIs) in wheelchair users, comprising:
providing a wheelchair with a containe wherein the container houses at least one urinary catheter drainage bag; at least one connector for removably attaching the container to a wheelchair; and wherein the container is positioned to maintain the drainage bag at an elevation below the user's bladder when seated in the wheelchair; and
using the container to maintain a urinary catheter drainage bag in a position that prevents urine backflow.

11. The method of claim 10, wherein the container is washable to maintain hygiene.

12. The method of claim 10, wherein the wheelchair includes multiple attachment points for positioning the container based on user preference and medical requirements.

13. The method of claim 10, wherein the system is utilized in a hospital or nursing home setting to improve patient care and reduce healthcare costs.

14. The method of claim 10, further incorporating an alert system to notify caregivers when the drainage bag needs attention or replacement.

15. The method of claim 10, further including steps for user training on the placement and removal of the drainage bag.

16. A privacy-enhancing urinary catheter drainage bag system for wheelchair users, comprising:
a container that provides concealment for at least one urinary catheter drainage bag;
attachment means for securing the container to a wheelchair;
wherein the system is designed to allow user access to the drainage bag without assistance.

17. The privacy-enhancing system of claim 16, further including an educational component provided to the user for proper use and maintenance.

18. The privacy-enhancing system of claim 16, wherein the attachment means include adjustable straps to accommodate different wheelchair models.

19. The privacy-enhancing system of claim 16, wherein the container includes a visually opaque material to ensure privacy.

20. The privacy-enhancing system of claim 16, wherein the system is part of a comprehensive patient care protocol aimed at reducing CAUTIs and enhancing patient dignity.
